(19) United States
(12) Patent Application Publication
(10) Pub. No.: US 2008/0138416 A1
(43) Pub. Date: Jun. 12, 2008

(54) METHOD AND SYSTEMS FOR USING BIOPOLYMER-BASED BEADS AND HYDROGELS

(75) Inventors: Francis Rauh, Plainsboro, NJ (US); Randall J. Lee, Hillsborough, CA (US); Mark Maciejewski, Edina, MN (US)

Correspondence Address: Patent Administrator, FMC Corporation, 1735 Market Street, Philadelphia, PA, 19103, US

(73) Assignee: FMC BIOPOLYMER AS, Tomtegt (NO)

(21) Appl. No.: 11/762,611

(22) Filed: Jun. 13, 2007

Related U.S. Application Data
(60) Provisional application No. 60/813,184, filed on Jun. 13, 2006.

(51) Int. Cl. A61K 9/16; A61K 31/738; A61K 31/734; A61K 38/06; A61K 38/07; A61K 38/08; A61K 38/10; A61K 35/12; C12N 5/02; A61P 43/00

(52) U.S. Cl. 424/488; 435/1.1; 424/93.21; 424/93.7; 514/18; 514/17; 514/15; 514/16; 514/14; 514/54; 424/493; 435/404

(57) ABSTRACT

Compositions comprising biopolymers such as alginates and cell attachment peptides are disclosed. Compositions may optionally further comprise cells. Methods for repairing or treating a tissues and organs with such compositions and systems for providing such compositions to tissues and organs, and methods for delivering desired proteins to individual with such compositions and systems for providing such compositions are also disclosed. In vitro methods of culturing cells are also disclosed.

### CROSS-REFERENCE TO RELATED APPLICATION

This application claims the benefit of U.S. Provisional Patent Application No. 60/813,184 filed Jun. 13, 2006, which hereby is incorporated herein in its entirety by reference thereto.

### FIELD OF THE INVENTION

The present invention relates biopolymer beads and hydrogels and methods and apparatus for using such biopolymer beads and hydrogels for treating individuals and repairing tissue.

### BACKGROUND OF THE INVENTION

U.S. Pat. Nos. 4,988,621, 4,792,525, 5,965,997, 4,879,237, 4,789,734 disclose cell attachment peptides which are biologically active molecules for cell adhesion or other cellular interaction. Cells attach to such peptides. Other cell attachment peptides are also known including those that bind to some cell types and not others.

The uses of biopolymer hydrogels linked to cell attachment peptides in implantable compositions which may comprise cells are disclosed in U.S. Pat. No. 6,642,363. Biopolymer matrices are particularly useful due to the high degree of biocompatibility of the materials used, particularly alginates, chitosan, hyaluronan and compositions comprising mixtures thereof.

There remains a need for improved compositions and uses of compositions comprising biopolymers linked to cell attachment peptides.

### BRIEF SUMMARY OF THE INVENTION

Biopolymer beads and hydrogels are provided. Such biopolymer beads and hydrogel compositions may be used in the treatment of various diseases and conditions. In some embodiments, the biopolymer beads and hydrogels are implanted with or without various cell types. In some embodiments, biopolymer beads and hydrogels comprising alginate polymers bonded to peptides are provided. In some embodiments, biopolymer beads are provided comprising a core in which peptides are dispersed with alginate polymers, and a chitosan film ionically bonded to available alginate polymers at the surface of the core. In some embodiments, biopolymer beads are provided comprising a core in which peptides and chitosan derivates are dispersed with alginate polymers and form alginate-peptide complexes to which the chitosan derivatives are bonded. In some embodiments, biopolymer beads are provided comprising a core of chitosan polymers which may or may not be bonded to peptides.

In some embodiments, diseases and conditions are treated by implanting biopolymer beads or hydrogels that comprise an agent comprising one or more materials having cell-recruiting and/or angiogenic-initiating properties.

Compositions are provided which are selected from the group consisting of: compositions comprising alginate covalently linked to two or more different cell attachment peptides; compositions comprising alginate mixed with two or more different cell attachment peptides; compositions comprising alginate covalently linked to one or more different cell attachment peptides and mixed with one or more different cell attachment peptides; compositions comprising alginate covalently linked to one cell attachment peptides and ionically linked to chitosan; compositions comprising alginate covalently linked to two or more different cell attachment peptides and ionically linked to chitosan; compositions comprising alginate mixed with one cell attachment peptides and ionically linked to chitosan; compositions comprising alginate mixed with two or more different cell attachment peptides and ionically linked to chitosan compositions comprising alginate covalently linked to one or more cell attachment peptide and mixed with one or more cell attachment peptides and ionically linked to chitosan; compositions comprising alginate covalently linked to two or more different cell attachment peptide and mixed with different cell attachment peptides and ionically linked to chitosan; compositions comprising cells encapsulated in alginate covalently linked to two or more different cell attachment peptides; compositions comprising cells encapsulated in alginate mixed with two or more different cell attachment peptides; compositions comprising cells encapsulated in alginate covalently linked to one or more different cell attachment peptides and mixed with one or more different cell attachment peptides; compositions comprising cells encapsulated in alginate covalently linked to one cell attachment peptides and ionically linked to chitosan; compositions comprising cells encapsulated in alginate covalently linked to two or more different cell attachment peptides and ionically linked to chitosan; compositions comprising cells encapsulated in alginate mixed with one cell attachment peptides and ionically linked to chitosan; compositions comprising cells encapsulated in alginate mixed with two or more different cell attachment peptides and ionically linked to chitosan compositions comprising cells encapsulated in alginate covalently linked to one different cell attachment peptide and mixed with different cell attachment peptides and ionically linked to chitosan; and compositions comprising cells encapsulated in alginate covalently linked to two or more different cell attachment peptide and mixed with different cell attachment peptides and ionically linked to chitosan.

Methods for repairing or treating a tissue or organ are provided. The methods comprise providing to the tissue or organ a therapeutically effective amount of such compositions.

Systems for repairing or treating tissues or organs are provided. Some systems comprise a therapeutically effective amount of such compositions and a delivery device for providing the composition to a tissue or organ.

Some systems comprise a delivery device for applying two or more components to a tissue or organ. In some such systems, the first component comprises a gelling ion and the second component is selected from the group consisting of one or more of the following: a composition comprising non-crosslinked alginate covalently linked to two or more different cell attachment peptides, and optionally further comprising cells; a composition comprising non-crosslinked alginate mixed with two or more different cell attachment peptides and optionally further comprising cells; and a composition comprising non-crosslinked alginate covalently linked to one or more different cell attachment peptides and mixed with one or more different cell attachment peptides and optionally further comprising cells.

In some systems comprising a delivery device for applying two or more components to a tissue or organ, the first component comprises a mixture of gelling ions and one or more different cell attachment peptides; and the second component comprising a therapeutically effective amount of selected from the group consisting of one or more of the following: a composition comprising non-crosslinked alginate, and optionally further comprising cells; a composition comprising non-crosslinked alginate covalently linked to one or more different cell attachment peptides, and optionally further comprising cells; a composition comprising non-crosslinked alginate mixed with one or more different cell attachment peptides and optionally further comprising cells; and a composition comprising non-crosslinked alginate covalently linked to one or more different cell attachment peptides and mixed with one or more different cell attachment peptides and optionally further comprising cells; wherein the first component and the second component collectively comprise two or more different cell attachment peptides.

Methods for delivering desired proteins to individuals are provided. The methods comprise providing to the individual a therapeutically effective amount of such compositions which comprise cells that express the desired protein.

Systems delivering desired proteins to individuals are provided.

Methods of culturing cells in vitro are also provided. The methods comprise maintaining cells under conditions suitable for cell growth and proliferation in composition provided herein.

### BRIEF DESCRIPTION OF THE DRAWINGS

FIG. 1A is a schematic cross-sectional view of a biopolymer bead with an alginate core material with a covalently attached peptide moiety.

FIG. 1B is a schematic cross-sectional view of the biopolymer bead depicted in FIG. 1A with a chitosan biopolymer overcoat.

FIG. 1C is a schematic cross-sectional view of a biopolymer bead with a core material containing an alginate:peptide complex with ionically attached low molecular weight chitosan and the core surface overcoated with high molecular weight chitosan.

### DESCRIPTION OF EMBODIMENTS OF THE INVENTION

The various methods, apparatus and materials described herein are suitable for use in tissue and organ repair and reconstruction. Various biopolymer-based bead agents and hydrogels are described which may be injected into tissue or organs either alone or with other material to provide therapeutic support or tissue engineering scaffold within tissue and organ structures, or to induce angiogenesis, or to recruit cells, or to prevent apoptosis to expedite tissue or organ repair/reconstruction. Such biopolymer-based beads and hydrogels further comprise, either attached covalently, or in a mixture therewith, two or more different biologically active molecules for cell adhesion or other cellular interaction. Combinations of two or more different cell attachment peptides linked to and/or mixed with biopolymer beads or gels provide particularly useful advantages for repairing, reconstructing and treating conditions of tissues and organs.

Biologically active molecules for cell adhesion or other cellular interaction are well known and widely recognized and available. U.S. Pat. Nos. 4,988,621, 4,792,525, 5,965,997, 4,879,237, 4,789,734 and 6,642,363, which are incorporated herein by reference, disclose numerous examples. Suitable peptides include, but are not limited to, peptides having about 10 amino acids or less. In some embodiments, cell attachment peptides comprise RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). In some embodiments, cell attachment peptides comprise RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22) and further comprise additional amino acids, such as for example, 1-10 additional amino acids, including but not limited 1-10 G residues at the N or C terminal. Cell attachment peptides comprising the RGD motif may be in some embodiments, 3, 4, 5, 6, 7, 8, 9 or 10 amino acids in length. Examples include, but are not limited to, RGD, GRGDS (SEQ ID NO:6), RGDV (SEQ ID NO:7), RGDS (SEQ ID NO:11), RGDF (SEQ ID NO:12), GRGDY (SEQ ID NO:17), GRGDSP (SEQ ID NO:18), GGGGRGDSP (SEQ ID NO:20) and GGGGRGDY (SEQ ID NO:21). In some embodiments, cell attachment peptides consist of RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). Biologically active molecules for cell adhesion or other cellular interaction may include EGF, VEGF, b-FGF, FGF, TGF, TGF-β or proteoglycans.

In some embodiments, beads and hydrogels include at least one cell attachment peptide that comprises the RGD motif. In some embodiments, beads and hydrogels include two or more different cell attachment peptides that comprise the RGD motif. In some embodiments, beads and hydrogels include at least one cell attachment peptide that does not comprise the RGD motif. In some embodiments, beads and hydrogels include two or more different cell attachment peptides that do not comprise the RGD motif.

In some embodiments, cell attachment peptides are covalently linked to alginate polymers. When referring cell attachment peptides and alginate polymers it is understood that there are two different numbers used in referring to the peptides: in one instance the number refers to the number of peptide molecules per alginate molecule; in the second instance the number refers to the number of different peptides, i.e. peptides with different sequences amino acid sequences. This, reference to “alginate covalently linked to two or more different cell attachment peptides is intended to mean multiple alginate polymer molecules to which at least one alginate polymer molecule is covalently linked to at least one cell attachment peptide and at least one other alginate polymer molecule covalently linked to at least one different cell attachment peptide. In some embodiments, it refers to multiple copies of a cell attachment peptide attached to a single alginate molecule. In some embodiments, it refers to one or more single alginate molecules that contain two different cell attachment peptides covalently linked to it. In some embodiments, one or more single alginate molecule that contain multiple copies of two different cell attachment peptides covalently linked to it. In some embodiments, it refers to a mixture comprising a plurality of alginate molecules that contain one or more cell attachment peptides covalently linked to it and a plurality of other alginate molecules that contain one or more different cell attachment peptides covalently linked to it. In some embodiments, it refers to a mixture comprising a plurality of alginate molecules that contain multiple copies of one or more cell attachment peptides covalently linked to it and a plurality of other alginate molecules that contain multiple copies of one or more different cell attachment peptides covalently linked to it. In addition to all the various combinations of multiple copies of peptides on alginate molecules and different and the same peptides and mixtures thereof, “alginate covalently linked to two or more different cell attachment peptides is also intended to mean all such various combinations mixed with alginate molecules that are free of cell attachment peptides.

Accordingly, when referring to “alginate mixed with two or more different cell attachment peptides” each alginate polymer molecule may contain a single copy or multiple copies of one or more cell attachment peptide provided the collective plurality of alginate polymer molecules include two or more different cell attachment peptides, i.e. peptides with different sequences. Similarly, when referring to “alginate covalently linked to one or more cell attachment peptides” each alginate polymer molecule may contain a single copy or multiple copies of cell attachment peptide having the same sequences or different sequences. When referring to “alginate covalently linked to one cell attachment peptides” each alginate polymer molecule may contain a single copy or multiple copies of a cell attachment peptide, and may optionally contain others.

In some embodiments, 1-20 individual cell attachment peptides are covalently linked to each alginate polymer. In some embodiments, cell attachment peptide is identical. In some embodiments, two or more different cell attachment peptides are covalently linked to each alginate polymer. In some embodiments, beads and hydrogels are made with a mixture of alginate polymers having different types or different numbers of cell attachment peptides covalently linked to alginate polymers. Further, beads and hydrogels may be made with a mixture of alginate polymers having cell attachment peptides covalently linked to alginate polymers and alginate polymers free of peptide attachment peptides.

The biopolymer-based beads and hydrogels may contain only biopolymer material and peptides, or they may include cells such as stem cells. In some embodiments, cells deriving from the mesoderm, endoderm, ectoderm or the neural crest. In some embodiments cells are selected from the group consisting of: muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins. In addition, biopolymer-based beads and hydrogels in combination with peptides, with or without cells, may also contain proteins, plasmids, or genes; growth factors in either protein or plasmid form; chemo-attractants; fibrin factor (or fragment) E; various pharmaceutical compositions; neo-tissues; or other therapeutically beneficial materials; or any combination of the foregoing.

Suitable biopolymers include alginates, chitosan, hyaluron, fibrin glue, and collagen. The biopolymer or combination of biopolymers and other material may be fabricated as beads or as hydrogels. Various techniques may be used to limit migration or diffusion of the beads and hydrogels from the site of injection. In one technique, beads may be introduced with a biopolymer anchoring component such as fibrin glue or chitosan. In another technique, beads may contain matrix-forming material such as fibrin glue components encapsulated in rapidly biodegradable material so as to be rapidly released to form an in situ matrix. In another technique, beads may be provided with an adhering material at the surface for adhering to tissue. The adhering material may be formulated so that the beads do not adhere to one another within the delivery system. The beads may be coated with a suitable material so as not to interact with one another within the delivery system, or to provide a controlled-release property. Also, the rate of resorption of the biopolymer system may be controlled by varying the degree of cross linking and the molecular weight of the components using any suitable technique, one illustrative technique being described in, for example, Kong, et al “Controlling rigidity and degradation of alginate hydrogels via molecular weight distribution,” Biomacromolecules, 2004, 5, 1720-1727, which is incorporated herein by reference. In another study, cross-linking in an alginate solution was achieved by adding 2.5 millimolar of Ca²⁺ per gram of alginate resulting in a Young's Modulus of 12.3 Kilo Pascal (KPa) for the resulting film measured via stress-relaxation testing. Furthermore, at a higher spiking concentration of 62.5 millimolar of Ca²⁺ per gram of alginate resulted in a Modulus of 127 KPa. To achieve desired therapeutic results when injecting into human tissue, it may be desirable for the alginate solution to be in the range 0.1% to 2% weight/volume cross-linked alginate, wherein desirable injection volumes may be in the range of approximately 0.1 to 1.5 milliliters. In addition to the example cited above, cross-linking of alginate solutions may be accomplished with additional divalent cations such as Mg²⁺, Sr²⁺, or Ba²⁺.

Among other subject matter described herein are improved systems and methods, which may include improved compositions of matter, which advantageously are effective for achieving: treatment of diseases and disorders of tissues and organs including, but not limited to, bones, muscle, cartilage, connective tissue, nerve, epithelial, vascular, mucosal, sinus, skin, liver, lungs, kidneys, brain, pancreas, endocrine organs, esophagus, stomach, intestines, colon, and rectum. According to some embodiments, improved systems and methods, which may include improved compositions of matter, which advantageously are effective for achieving: treatment of diseases and disorders of tissues and organs including, but not limited to, compositions useful in the repair, reconstruction or treatment of conditions and diseases of soft tissue, nerve, liver, kidney, bone, cartilage, knee, shoulder, rotator cuff, ligaments and tendon, digestive tract (gut, stomach), and epithelial cells (cornea, breast, pancreas, urothelial). According to some embodiments, improved systems and methods, which may include improved compositions of matter, comprise: cells deriving from mesoderm, endoderm, ectoderm or the neural crest including but not limited to corneal cells; breast cells, including preadipocytes; liver cells; pancreatic islets; tracheal gland cells; kidney cells; gastrointestinal derived cells; urothelium cells; prostate cells, including prostatic epithelial and prostatic stromal cells; cervix cells; vaginal cells; adipose cells; smooth muscle cells; cardiac-derived muscle cells, including cardiac myocytes; skeletal muscle cells including satellite cells and fibroblasts; cartilage; bone cells, including osteoblasts and osteoclasts; blood vessel cells, including vascular endothelial and perivascular endothelial cells; endocrine cells; glia and neurons, including Schwann cells, olifactory ensheathing cells, hippocampal and spinal neurons; testis cells, including leydig sertoli and germ cells; ovarian cells, including granulose, follicles and germ cells; pluripotent stem cells; neural stem cells; liver stem cells; muscle stem cells; endothelial progenitor cells; mesenchymal cells; chondrogenic stem cells; hematopoietic stem cells; and lymphoid cells including blood peripheral mononuclear cells.

Also described herein are improved systems and methods, which may include improved compositions of matter, which advantageously are effective for cell therapy and tissue engineering and achieving: treatment of diseases and disorders through the introduction of cells into a patient's body. Such cells include stem cells. In some embodiments, cells are derived from the mesoderm, endoderm, ectoderm or the neural crest. In some embodiments cells are selected from the group consisting of: muscle cells, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins, muscle cells, chondrocytes, hepatic cells, neural cells, renal cortex cells, vascular endothelial cells, thyroid and parathyroid cells, adrenal cells, thymic cells, ovarian cells, and other cell types of primary origin and cells which include recombinant genetic material provided to express desired proteins, such as for example a cytokine, a growth factor, insulin, factor VIII, factor IX, or an angiogenesis inhibitor such as angiostatin or endostatin.

Methods for delivering a desired protein to an individual comprising: providing to the individual a therapeutically effective amount of a composition selected from the group consisting of one or more of the following: a composition comprising cells encapsulated in alginate covalently linked to two or more different cell attachment peptides; a composition comprising cells encapsulated in alginate mixed with two or more different cell attachment peptides; a composition comprising cells encapsulated in alginate covalently linked to one or more cell attachment peptides and mixed with one or more cell attachment peptides; a composition comprising cells encapsulated in alginate covalently linked to one cell attachment peptides and ionically linked to chitosan; a composition comprising cells encapsulated in alginate covalently linked to two or more different cell attachment peptides and ionically linked to chitosan; a composition comprising cells encapsulated in alginate mixed with one cell attachment peptides and ionically linked to chitosan; a composition comprising cells encapsulated in alginate mixed with two or more different cell attachment peptides and ionically linked to chitosan; a composition comprising cells encapsulated in alginate covalently linked to one cell attachment peptide and mixed with cell attachment peptides and ionically linked to chitosan; and a composition comprising cells encapsulated in alginate covalently linked to two or more different cell attachment peptide and mixed with cell attachment peptides and ionically linked to chitosan; wherein said cells express the desired protein The individual may have been diagnosed with a disease or condition characterized by an absence or deficiency in a functional form of the desired protein. Examples include, but are not limited to, diabetes, hemophilia, liver disease, and endocrine diseases. The individual may have been diagnosed with a disease or condition in which the desired protein may be a therapeutically beneficial protein including but not limited to those in which the individual may produce the desired protein naturally but which may be preferentially be supplemented with additional cells that express the protein.

Some of these systems and methods, which may include improved compositions of matter, involve a scaffold within tissue structures for enhanced retention and viability of implanted cells within tissue structures; an injectable scaffolding agent for injection into structures; injection of therapeutic, internal wall scaffolding within structures; and therapeutic mechanical scaffolding within a structure as an internal wall support.

Other of these systems and methods, which may include improved compositions of matter, involve therapeutic angiogenesis to transplanted cells within a patient; angiogenesis into tissue structures, including those receiving cell implant therapy; inducement or enhancement of therapeutic angiogenesis in tissue and organ structures or in injected structure scaffolds; and inducement of angiogenesis in a tissue or organ structure at least in part with an injected polymer agent.

Other of these systems and methods, which may include improved compositions of matter, involve enhanced retention of transplanted cells in a patient; enhanced retention and viability of implanted cells within tissue or organ structures; retention of living cells in a therapeutic mechanical scaffolding within a tissue or organ structure by use of an injectable combination of such living cells with a polymer agent; enhanced deposition of cells into a tissue or organ structure of a patient; and an induced deposition of autologous cells within a tissue or organ structure of the patient at least in part with an injected polymer agent.

Other of these systems and methods, which may include improved compositions of matter, involve additional cellular recruitment and deposition into tissue or organ structures receiving cell implant therapy; and use of factors adapted to recruit endogenous cells, including providing a cellular deposition recruiting factor.

It is to be appreciated that these systems and methods may be used individually or in various combinations with one another, and may involve more detailed aspects which may also be beneficial with respect to achieving the technological and other effects of one or more of the preceding aspects, or otherwise providing other substantial benefits.

Reference is made herein to providing scaffolding in tissue and organs, generally sufficient to achieve therapeutic result to damaged tissue. It is to be appreciated that such terms as “support” and “scaffold” are intended to mean, in one regard, that a significant result of the intervention is providing a mechanically relevant, structural improvement, which may be with regard to one structural aspect or several. The structural improvement may be of varying degrees, ranging from rigid to compliant, and may be achieved by various mechanisms, including matrices as well as unlinked particles imbedded in interstitial regions of the tissue or organ. In a similar regard, at some level it may be the case that most materials have some injectability and some scaffolding features too many if not most types of cells. However, a material is herein considered substantially an injectable scaffolding material with respect to cells if such material causes measurable benefit, and furthermore in most circumstances that is not outweighed by more deleterious detriment. Moreover, it is also contemplated that while chronically improved support to damaged tissue has been observed, such chronic results may not be required to gain value and benefit from treatment in all cases

Agents such as biopolymer-based bead agents, chitosan hydrogel-based agents, alginate hydrogel-based agents, and other agents may be used. In some embodiments, alginate polymers of an alginate matrix contain more than 50% α-L-guluronic acid. In some embodiments, the alginate polymers of the alginate matrix contain more than 60% α-L-guluronic acid. In some embodiments, the alginate polymers of the alginate matrix contain 60% to 80% α-L-guluronic acid. In some embodiments, the alginate polymers of the alginate matrix contain 65% to 75% α-L-guluronic acid. In some embodiments, the alginate polymers of the alginate matrix contain more than 70% α-L-guluronic acid. In some embodiments, the alginate polymers of the alginate matrix have an average molecule weight of from 20 to 500 kD. In some embodiments, the alginate polymers of the alginate matrix have an average molecule weight of from 50 to 500 kD. In some embodiments, the alginate polymers of the alginate matrix have an average molecule weight of from 100 to 500 kD.

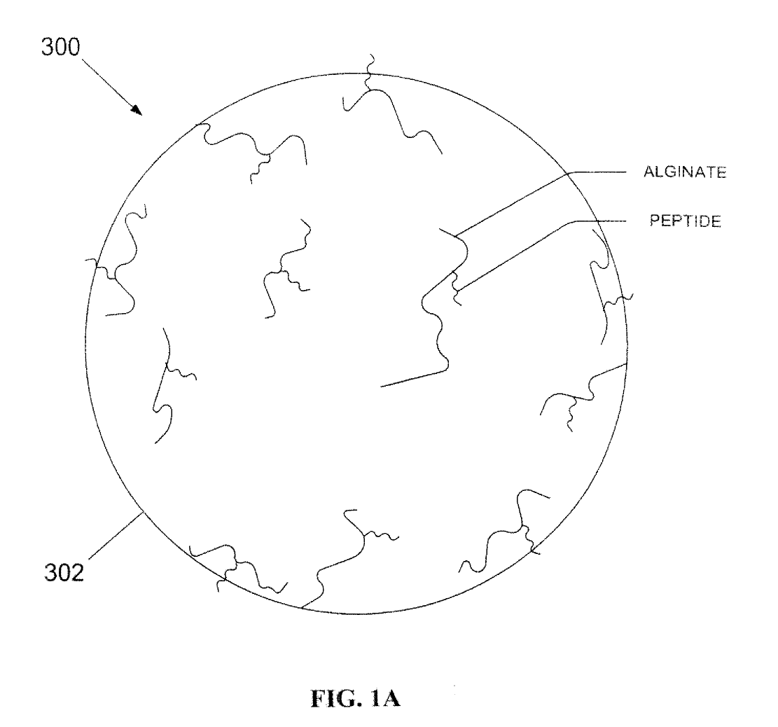

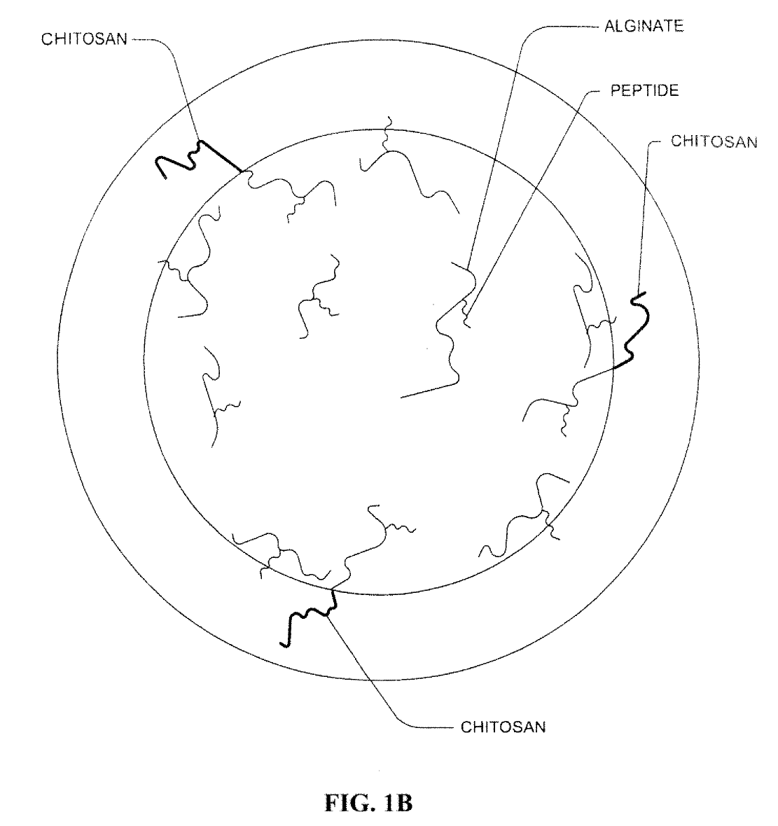

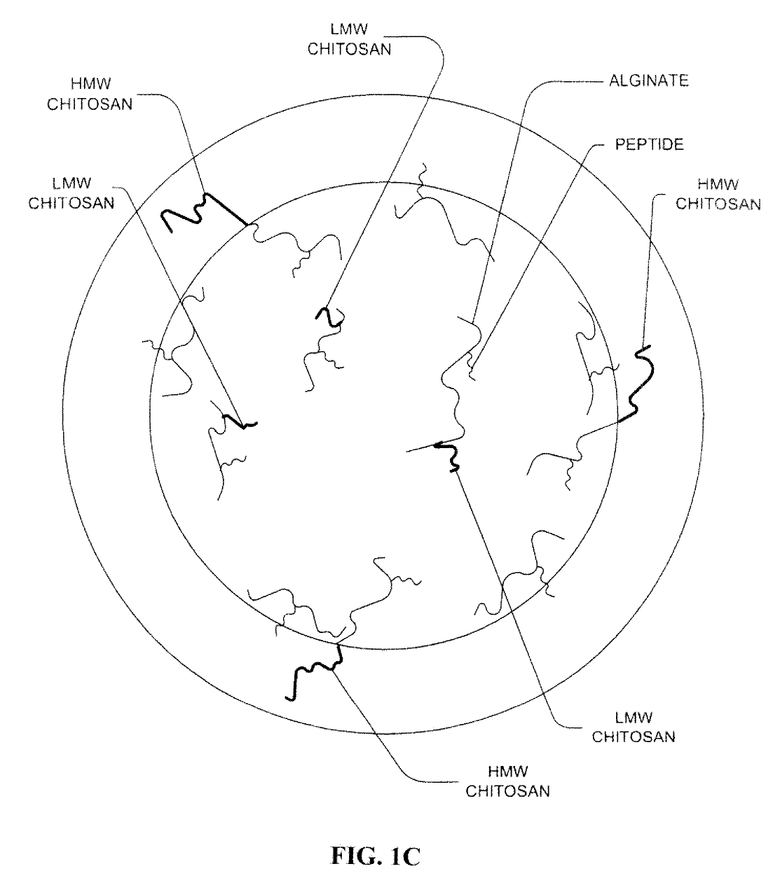

A cross-sectional schematic representation of a biopolymer bead 300 is shown in FIG. 1A. The bead 300 may have a geometrical core 302 of alginate type material. The bead core 302 surface geometry may be spherical, elliptical, out of round, and/or contain surface irregularities. The term bead as used herein is intended to encompass all of the aforementioned geometries.

The bead core 302 may, if desired, have cell attachment peptides moieties covalently bonded to the alginate polymer. In some embodiments, two different cell attachment peptides moieties covalently bonded to the alginate polymer. In some embodiments, one cell attachment peptide moiety is covalently bonded to some alginate polymers and a different cell attachment peptide moiety is covalently bonded to different alginate polymers and the two are mixed together. In some embodiments, one cell attachment peptide moiety is covalently bonded to alginate polymers and a different cell attachment peptide moiety is mixed with alginate polymers.

Peptide synthesis services are available from numerous companies, including Commonwealth Biotechnologies, Inc. of Richmond, Va., USA. Chemical techniques for coupling peptides to the alginate polymer backbones may be found in U.S. Pat. No. 6,642,363 issued Nov. 4, 2003 to Mooney et al., which is incorporated herein by reference. In addition to having peptides dispersed throughout the core region of the bead, it may be advantageous to have specific cell attachment peptides (for example RGD and/or GREDVY) exposed on the surface of the bead and in sufficient concentration to enhance anchoring to underling tissue. To increase the surface concentration of cell attachment peptides, the beads may be dip coated or spray coated with a solution/mist containing the peptide chemistry to ensure all available potential alginate bonding sites on the surface are saturated with cell attachment peptides.

The bead core 302 may be manufactured by means well known in the field of microencapsulation. One such technique is electrostatic bead generation, which is particularly suitable for manufacturing beads as small as about 200 μm. In this technique, a solution containing dissolved alginate material is injected into a needle oriented vertical, aimed downward. Directly below the needle tip, displaced a predetermined distance (the dropping distance) is placed a capturing aqueous solution. An electrostatic potential of typically a few kilovolts is applied between the needle tip and the capturing aqueous solution to pull the droplets from the needle tip. The individual droplets are then harvested one-by-one as they fall into the capturing aqueous solution. The size of the beads can be controlled by varying any of the following variables: the inside diameter of the needle tip, the magnitude of the electrostatic potential, the concentration of alginate in solution, the dropping distance, and combinations thereof. One such commercially available instrument to manufacture alginate beads as explained above is the Nisco Engineering Encapsulation Unit Type V1, which is available from Nisco Engineering AG, Dufourstrasse 110, CH-8008 Zurich, Switzerland, and which is described in a document included within this application as an appendix. Also, the alginate core material may, or may not, have one or more different peptide moieties covalently attached to the alginate biopolymer prior to bead fabrication as explained above. An overview of peptide chemical attachment to alginate polymers may be found in U.S. Pat. No. 6,642,363 issued Nov. 4, 2003 to Mooney et al., which is incorporated herein by reference.

In some embodiments, alginate formulations have certain angiogenic properties and the peptides have been known to have cell signaling properties, i.e., attracting stem cells amongst other cellular types to the area of injection.

In cases where it may be desired to anchor the bead(s) to the immediate area of injection, it may be desirable to overcoat the alginate bead with a coating both chemically attached to the alginate surface on the inboard side of the coating and simultaneously bonded to tissue on the outboard. Given that both the alginate surface and the tissue have negative bonding sites available, an overcoat with a positive charge density may be appropriate. Chitosan is such a material. Chitosan and its derivatives are biopolymer materials used in a wide range of medical applications. Chitosan is a linear polysaccharide, and given its positive charge density is a bioadhesive which readily binds to negatively charged surfaces such as mucosal membranes. FIG. 1B is a schematic representation of an alginate core/chitosan overcoat bead. The alginate core may be manufactured by the technique describe above or by any known equivalent to those skilled in the art of micro encapsulation. The chitosan overcoat may be applied by dip coating or other known procedures, wherein the chitosan may ionically bond to the available negative sites on the alginate surface. Given this, the chitosan may act as an anchor to immobilize the beads to the negatively charged tissue, giving temporary mechanical integrity to the MI damaged tissue. Temporary, in the sense that the chitosan overcoat will eventually be enzymatically dissolved. “Anchoring time” may be prolonged by increasing the thickness of the chitosan overcoat.

An alternative approach to increasing the “anchoring time” without relying solely on increasing the chitosan overcoat thickness is depicted in FIG. 1C. An alginate core, with or without covalently attached peptide, may be manufactured by the electrostatic bead generation technique described earlier. The alginate core may then be dip coated in a solution containing a mixture of both low and high molecular weight chitosan derivatives. The low molecular weight chitosan derivatives may be sufficiently small and have sufficient kinetic energy to tunnel through voids available in the alginate core surface and diffuse into the core region ultimately encountering an ionic bond with an inner core alginate. Upon completion of the dip coat, the now alginate:chitosan impregnated core may have an overcoat consisting of a mixture of both high and low molecular weight chitosan. However, when now dissolved down to and into the core, there may be a sufficient population of chitosan polymers (ionically bonded to core alginates) and with sufficient positive charge sites left available to prolong the anchoring process while the core itself is biodegrading away.

Since manufacturing techniques such as the electrostatic technique are capable of making very large beads on the order of a few millimeters, the upper bead size limit depends on a number of practical factors other than the manufacturing technique. Bead sizes in excess of 500 μm and with good adhesion properties may be suitable for direct injection into damaged tissue, provided the beads do not encapsulate living cells. However, if living cells are to be encapsulated, the upper size limit may be dictated by diffusion limitations of nutrients such as oxygen for the encapsulated cells, with beads on the order of 500 μm or less being typical. For the alginate and/or chitosan encapsulation of cells, proteins, or other biological materials using known bead generation techniques, for example, an appropriate size range of the beads for direct injection into damaged tissue is from about 30 μm to about 500 μm.

Generally, it is desired to match delivery of cells and other scaffolding closely to the damaged area, so that the delivery syringe or catheter desired to achieve a dispersed injection would be suitably adapted to inject the scaffolding material along a predetermined expansive and shaped region. Such custom delivery and resulting scaffolding provides for reliable and controlled impact of the therapy. In other words, “contacting” a region of tissue is considered contextual to the particular embodiment or application, and may be substantially continuous and uninterrupted contact in certain circumstances, or in others may have interruptions that are considered insignificant in the context of the anatomy or more general use.

In further exemplary modifications, needles may be replaced by other modes for delivering the desired agent, such as through walls of porous membranes adapted to be engaged against tissue for delivery. In still further embodiments, those particular embodiments described above for injecting scaffolding within tissue may also be combined with various devices, structures, and techniques.

Different volumes of scaffolding agent, and different numbers, sizes, patterns, and/or lengths of injection needles may be used to suit a particular need. In one regard, a prior diagnostic analysis may be used to determine the extent of the condition, location of the condition, or various anatomical considerations of the patient which parameters set forth the volume and/or pattern of scaffold agent or injection needle array to use for delivery. Or, a real time diagnostic approach may allow for stimulus or other effects to be monitored or mapped, such that the amount of agent, or distance, direction, or number of needle deployment, is modified until the correct result is achieved. Therefore, for example, the needles of such embodiments may be retractable and advanceable through tissue so that different arrangements may be tried until the damaged region is mapped and characterized for appropriate scaffolding injection.

### Beads and Hydrogels Having Therapeutic Properties

A variety of biological material may be delivered with injectable biopolymer-based beads and hydrogels, including cells, proteins, plasmids, or genes; growth factors in either protein or plasmid form; chemo-attractants; fibrin fragment E; various pharmaceutical compositions; or other therapeutically beneficial materials; or any combination of the foregoing. The beneficial combination of two or more different cell attachment peptides such as RDG peptides or other cellular affinity factors, and fragment E (or other angiogenic factors), for example, may be achieved with beads and hydrogels.

Beads and hydrogels may be made to encapsulate cells in the following manner. In some embodiments, calcium alginate polymers that can form ionic hydrogels may be sufficiently malleable to be used to encapsulate cells. The hydrogel is produced by cross-linking the anionic salt of alginic acid, a carbohydrate polymer isolated from seaweed, with calcium cations, whose strength increases with either increasing concentrations of calcium ions or alginate. The alginate solution may then be mixed with the cells to be implanted to form an alginate suspension. The suspension may then be injected directly into a patient prior to hardening of the suspension. The suspension may then harden over a short period of time due to the presence in vivo of physiological concentrations of calcium ions. Specific examples of formulations to form ionic hydrogels from calcium alginate polymers may be found in U.S. Pat. No. 6,281,015 issued Aug. 28, 2001 to Mooney et. al., which is incorporated herein by reference. In some embodiments, two or more different cell attachment peptide moieties (e.g., RGD or GREDVY) may be mixed in solution with the alginic acid allowing covalent bonding between the peptides and the alginates prior to mixing with the cells to be injected. In some embodiments, one or more different cell attachment peptide moieties (e.g., RGD or GREDVY) may be mixed in solution with the alginic acid allowing covalent bonding between the peptides and the alginates prior to mixing with the cells and one or more different cell attachment peptides to be injected. In some embodiments, two or more different cell attachment peptide moieties (e.g., RGD or GREDVY) may be mixed in solution with the cells to be injected.

In an alternative embodiment, alginate or chitosan beads may encapsulate cells which have previously been ionically entrapped by nanoparticles. The procedure for encapsulation may include the electrostatic bead generation method and apparatus mentioned earlier or the coaxial air driven microencapsulator apparatus as discussed in documents by Nisco Engineering AG entitled “Micro-Encapsulators/Immobilisators/Granulators” and “Encapsulation Unit—Var JI,” available from Nisco Engineering AG, Dufourstrasse 110, CH-8008 Zurich, Switzerland, which are included within this application as an appendix. In another technique, alginate or chitosan beads may encapsulate cells dispersed in solution by way of a lypholizing (freeze drying) procedure utilizing a sufficient vacuum to crystallize the solution and entrap the cells. In this environment the freeze-dried beads may be temporarily packaged for shipment to a destination for their ultimate medical use wherein the beads may be re-hydrated prior to injection via hypodermic needle or air gun mist. In yet another technique, alginate beads may encapsulate cells by an emulsification/gelation process wherein an alginate solution containing an insoluble calcium salt is dispersed in oil, and gelation may be achieved by gentle acidification with an oil-soluble acid that causes calcium ion release. Specific examples of formulations to form alginate beads via the emulsification/gelation procedure may be found in published article “Microencapsulation of Hemoglobin in Chitosan-coated Alginate Microspheres Prepared by Emulsification/Internal Gelation,” AAPS Journal 2006, Vol 7. No. 4, Article 88, Jan. 13, 2006, by authors Caterina M. Silva et. al., which is incorporated herein by reference. Microspheres with a mean diameter of less than 30 μm and an encapsulation efficiency of above 90 percent are attainable with this technique.

Other suitable materials having beneficial effects in such combination are also contemplated, such as other polymers or molecular scaffolds or materials that intervene sufficiently to inter-cellular gap junctions or otherwise impact the extracellular matrix in cardiac tissue structures to substantially enhance function and/or support of a damaged wall structure. Moreover, collagen or precursors or analogs or derivatives thereof are further considered useful for this purpose, either in addition or in the alternative to fibrin glue.

Beads and hydrogels may contain or may be injected along with other materials, such as fluids or other substrates to provide the cells in an overall preparation as a cellular media that is adapted to be injected, such as in particular through a delivery lumen of a delivery catheter.

Beads and hydrogels may contain or be injected with other synthetic polymers, such as polyethylene oxide (“PEO”), PEO-poly-1-lactic acid (“PLLA-PEO block copolymer”), poly(N-isopropylacrylamide-co-acrylic acid) (“poly(NIPAAm-co-Aac)”), pluronics, and poly-(N-vinyl-2-pyrrolidone) (“PVP”).

Beads and hydrogels may be passivated with a coating such as sugar or a biopolymer, which is broken down when the beads are in situ in the heart by action of the body or by the use of an initiator combined and introduced with the passivated beads, or introduced into the same cardiac region as the passivated beads. Upon removal of the passivation coating, the surfaces of the beads are exposed so that the therapeutic effect of the beads may be realized.

Combining Beads and Hydrogels with Other Scaffolding Materials

Among the various embodiments an injectable material is described that is adapted to form a therapeutic scaffolding in tissue structures. Beads and hydrogels which comprise two or more different cell attachment peptides may be embedded within the therapeutic scaffolding and released as the scaffolding is adsorbed. Examples of highly beneficial materials for use according to the invention include: cells, polymers, or other fluids or preparations that provide interstitial or other forms of internal wall support, such as stiffening inter-cellular junction areas. Fibrin glue agent has been identified as a highly beneficial biopolymer for such use. Another example includes an injectable material containing collagen, or a precursor or analog or derivative thereof.

Therapeutically effective scaffolding may be made from fibrin glue. Fibrin glue is an FDA approved biomaterial that is routinely used as a surgical adhesive and sealant. This biopolymer is formed by the addition of thrombin to fibrinogen. Thrombin in a kit is an initiator or catalyst which enzymatically cleaves fibrinogen which alters the charge and conformation of the molecule, forming a fibrin monomer. The fibrin monomers then proceed to aggregate forming the biopolymer fibrin. After combination of the two thrombin and fibrinogen components, the solution remains liquid for several seconds before polymerizing. Fibrin glue agent, either immediately following mixture of the precursor materials, or by delivering the materials separately to mix in-situ, is therefore adapted to be delivered to the myocardium via injection catheters or other injectors, thus requiring only a minimally invasive procedure. It is also biocompatible and non-toxic, without inducing inflammation, foreign body reactions, tissue necrosis or extensive fibrosis.

As a support, fibrin glue may be modified to tailor its mechanical properties for this particular application. An increase in thrombin or fibrinogen concentration results in an increase in tensile strength and Young's modulus. An increase in fibrinogen concentration will also decrease the degradation rate of the biopolymer.

Fibrin glue is observed to be generally biocompatible, non-toxic, and not generally observed to induce inflammation, foreign body reactions, tissue necrosis or extensive fibrosis. Another benefit of this injectable scaffold is that it is an already FDA approved material, which is routinely used as a surgical adhesive and sealant. Since it remains liquid before combination of its two components, it could also be delivered via catheter, thus requiring only a minimally invasive procedure in humans.

### Benefits of Beads and Hydrogels Embedded Within a Fibrin Glue Scaffold

Beads may be included in either the thrombin or fibrogen components of fibrin glue, or in both components. Depending on the type of beads and hydrogels, therapeutically beneficial results in addition to those provided by the fibrin glue scaffold alone may be realized. The beads may encapsulate cells, which protects the cells and improves cell survival during injection.

Some applications may benefit from prolonging the presence of the scaffold. Where the scaffold is fibrin, for example, the fibrin is resorbed by enzymatic and phagocytic pathways so that a fibrin scaffold may disappear on the order of four weeks post-injection, or so.

One approach is to encapsulate the two components of fibrin glue, or of a scaffolding agent having a biopolymer capable of cross-linking such as an alginate or alginate-containing material and a cross-linking initiator, and inject the beads with the fibrin glue. As the in situ scaffold biodegrades, the exposed beads also biodegrade, thereby releasing their material which in turn forms new scaffolding. Alternatively, a mixture of instantly biodegradable beads and more slowly biodegradable beads may be injected, so that the instantly biodegradable beads immediately release their material to form an initial scaffold that is maintained over time by materials from the more slowly deteriorating beads.

### Materials Described Herein Generally Illustrate Broader Classes of Materials

The materials described herein generally illustrate certain broader classes of materials, which classes may contribute additional alternatives as would be apparent to one of ordinary skill. Where a compound is herein identified in relation to one or more embodiments described herein, such as for example collagen or fibrin, precursors or analogs or derivatives thereof are further contemplated. For example, material structures that are metabolized or otherwise altered within the body to form such compound are contemplated. Or, combination materials that react to form such compound are also contemplated. Additional materials that are also contemplated are those which have molecular structures that vary insubstantial to that of such designated compounds, or otherwise have bioactivity substantially similar thereto with respect to the intended uses contemplated herein (e.g. removing or altering non-functional groups with respect to such bioactive function). Such group of compounds, and such precursors or analogs or derivatives thereof, is herein referred to as a “compound agent.” Similarly, reference herein to other forms of “agents”, such as for example “polymer agent” or “fibrin glue agent” may further include the actual final product, e.g. polymer or fibrin glue, respectively, or one or more respective precursor materials delivered together or in a coordinated manner to form the resulting material.

It is to be appreciated that where fibrin glue or related agents are herein described, it is further contemplated that other materials such as collagen, or precursors or analogs or derivatives thereof, may also be used in such circumstances, in particular relation to forming injected scaffolding, either alone or in combination with cells.

The term “protein” is intended to include a wide variety of proteins. Another example of a suitable protein is integrin, which has been observed to enhance cellular binding and thus may be injected into cardiac tissue structures to provide substantial benefit to cellular tissue formation and/or retention there. For further illustration, further particular embodiments may also include integrin in combination with cell delivery, and/or in combination with others of the non-living compounds herein described.

### Injectable Hydrogels

Injectable materials which include two or more different cell attachment peptides may be used to form alginate and chitosan hydrogels to supply mechanical integrity for interstitial scaffolding, to retain various other materials in place, and so forth. Alginate hydrogels may be formed using either or both G-rich and M-rich alginate materials in the presence of divalent cations such Ca²⁺, Ba²⁺, Mg²⁺, or Sr²⁺. Gelling occurs when the divalent cations take part in ionic binding between blocks in the polymer chain, giving rise to a 3 dimensional network. In one approach, a dual chamber syringe converging into a single lumen injection needle may be used to inject the mixed components of the alginate mixture to gel in-vivo. One component may be a sodium alginate fully solubilzed in an aqueous solution such as H₂0. The other component may be one of the divalent cations mentioned above dispersed (not dissolved) in solution. The compounds may be mixed in any suitable manner. Prior to injection, for example, a T-type adapter attached to the syringe may be set to provide mixing of the components and initiate the gelling action, and then set to allow the alginate mixture undergoing gelling to enter the lumen and to be injected into the cardiac tissue of interest. The alginate mixture may be injected immediately, or may be allowed to partially pre-cure in the syringe in order to increase the viscosity of the hydrogel prior to injection. In some instances, a pre-cured formulation may reduce the possibility that a less viscous hydrogel may diffuse or migrate away from the tissue area of interest after injection. In order to limit or minimize diffusion/migration away from the injection site, it would be beneficial to utilize alginate materials with molecular weights in excess of about 300,000. In another approach, the sodium alginate solution and dispersed cation may be pre-mixed in an external mixing chamber, and aspirated into a single lumen syringe from which it may be injected into the cardiac tissue of interest. In another approach, the sodium alginate solution may be pre-mixed with one or more different cell attachment peptides for covalent attachment of the peptide to the alginate prior to mixing with the divalent cations. In addition to providing mechanical integrity for interstitial scaffolding, alginate hydrogels with covalently attached peptides may enhance cell proliferation in MI damaged cardiac tissue. In one in-vitro study, human umbilical vein endothelial cells (HUVEC) were utilized over a 10 day gestation period to demonstrate this effect. In this study, GRGDSP peptide material was covalently attached to high molecular weight M-type alginate (MW 297,000) in a ratio of 12 peptides per alginate molecule. HUVEC cells were added to the alginate solution and the solution was caused to gel by addition of 102 millimolar CaCl₂. HUVEC cells were also added to a negative control high molecular weight alginate solution without peptide attachment and caused to gel via addition of calcium chloride as before. Both gels were measured for density at day one via an optical absorption measurement at 490 nanometers and again at day 10. The negative control alginate w/o peptide showed a marginal increase in absorption from 0.4 to approximately 0.42 absorption units at day 10 indicating a small increase in cell population, whereas the peptide attached alginate increased from 0.4 to 1.0 absorption units (a 2.5× increase) over the same time period. Given that optical absorption units (Absorbance) are logarithmic in nature a 2.5× enhancement is significant (10^{2.5}≈316). For optimum cell proliferation in human endothelial 1 tissue, the peptide to alginate ratio may require clinical investigation, however the above results demonstrate promising in-vitro feasibility.

Single injections of agent with a single lumen catheter are suitable for agents that are designed not to clog a single lumen, because of the speed of injection, lessening of trauma, and relative ease of injection. However, a multiple-lumen catheter may be used if desired to deliver a multiple-part agent such as a first solution containing alginate an agent and a second solution containing gelling ion. The parts of a multiple-part formulation may be provided contemporaneously or serially, depending on the properties of the formulation. Multiple single lumen catheters may be used if desired. The formulation and catheter or catheters may be provided in kit form, or as individual components of an injection system.

### In Vitro Uses

In addition to application to tissue and organs, the compositions provided herein are useful in the in vitro cultivation of cells. In particular, the combination of two or more different cell attachment peptides in the context of a biopolymer-based bead or hydrogel provides advantages in the cultivation of cells which include higher levels of viability, and induction and maintenance of differentiation.

Although this written description contains many details, these details should not be construed as limiting the scope of the invention as set forth in the following claims, but should instead been seen as merely providing illustrations of various embodiments of this invention. Therefore, it will be appreciated that the scope of the present invention fully encompasses many variations and modifications of the embodiments described herein. Embodiments that include a description of a single element are not to be limited to one and only one such element. All structural, chemical, and functional equivalents to the elements of the described embodiments are to be considered within the scope of the invention. Moreover, it is not necessary for an apparatus or method to address each and every problem sought to be solved by the present invention, for it to be encompassed by the present invention. It will be appreciated that the apparatus may vary as to configuration and as to details of the parts, and that the method may vary as to the specific steps and sequence, without departing from the present invention.

1. A method for repairing or treating a tissue or organ comprising:
providing to a tissue or organ a therapeutically effective amount of a composition selected from the group consisting of one or more of the following:
a composition comprising alginate covalently linked to two or more different cell attachment peptides;
a composition comprising alginate mixed with two or more different cell attachment peptides;
a composition comprising alginate covalently linked to one or more cell attachment peptides and mixed with one or more different cell attachment peptides;
a composition comprising alginate covalently linked to one cell attachment peptides and ionically linked to chitosan;
a composition comprising alginate covalently linked to two or more different cell attachment peptides and ionically linked to chitosan;
a composition comprising alginate mixed with one cell attachment peptides and ionically linked to chitosan;
a composition comprising alginate mixed with two or more different cell attachment peptides and ionically linked to chitosan
a composition comprising alginate covalently linked to one cell attachment peptide and mixed with cell attachment peptides and ionically linked to chitosan;
a composition comprising alginate covalently linked to two or more different cell attachment peptide and mixed with cell attachment peptides and ionically linked to chitosan;
a composition comprising cells encapsulated in alginate covalently linked to two or more different cell attachment peptides;
a composition comprising cells encapsulated in alginate mixed with two or more different cell attachment peptides;
a composition comprising cells encapsulated in alginate covalently linked to one or more cell attachment peptides and mixed with one or more cell attachment peptides;
a composition comprising cells encapsulated in alginate covalently linked to one cell attachment peptides and ionically linked to chitosan;
a composition comprising cells encapsulated in alginate covalently linked to two or more different cell attachment peptides and ionically linked to chitosan;
a composition comprising cells encapsulated in alginate mixed with one cell attachment peptides and ionically linked to chitosan;
a composition comprising cells encapsulated in alginate mixed with two or more different cell attachment peptides and ionically linked to chitosan
a composition comprising cells encapsulated in alginate covalently linked to one cell attachment peptide and mixed with cell attachment peptides and ionically linked to chitosan; and
a composition comprising cells encapsulated in alginate covalently linked to two or more different cell attachment peptide and mixed with cell attachment peptides and ionically linked to chitosan.

2. The method of claim 1 wherein the cell attachment peptides are selected from group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

3. The method of claim 1 wherein said composition comprises alginate beads.

4. The method of claim 3 wherein the beads have mean diameters in a range of from about 30 um to about 500 um.

5. The method of claim 1 wherein said composition comprises an alginate hydrogel.

6. The method of claim 1 wherein said composition comprises cells selected from the group consisting of: stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

7. The method of claim 1 wherein said composition further comprises one or more compounds selected from the group consisting of: EGF, VEGF, b-FGF, FGF, TGF, TGF-β and proteoglycans.

8. The method of claim 1 wherein said composition is provided to a tissue selected from the group consisting of: bones, muscle, cartilage, connective, neural, epithelial, vascular, urothelial and mucosal.

9. The method of claim 1 wherein said composition is provided to an organ selected from the group consisting of: skin, sinus, liver, lungs, kidney, brain, pancreas, endocrine organs, esophagus, stomach, intestines, colon, rectum, cornea, and breast.

10. The method of claim 1 wherein said composition is provided as a cross-linked matrix.

11. The method of claim 1 wherein said composition produces a matrix in situ following application to the tissue or organ.

12. A system for repairing or treating a tissue or organ comprising a delivery device for providing a composition to a tissue or organ and a therapeutically effective amount of a composition selected from the group consisting of one or more of the following:
a composition comprising alginate covalently linked to two or more different cell attachment peptides;
a composition comprising alginate mixed with two or more different cell attachment peptides;
a composition comprising alginate covalently linked to one or more cell attachment peptides and mixed with one or more cell attachment peptides;
a composition comprising alginate covalently linked to one cell attachment peptides and ionically linked to chitosan;
a composition comprising alginate covalently linked to two or more different cell attachment peptides and ionically linked to chitosan;
a composition comprising alginate mixed with one cell attachment peptides and ionically linked to chitosan;
a composition comprising alginate mixed with two or more different cell attachment peptides and ionically linked to chitosan
a composition comprising alginate covalently linked to one cell attachment peptide and mixed with cell attachment peptides and ionically linked to chitosan;
a composition comprising alginate covalently linked to two or more different cell attachment peptide and mixed with cell attachment peptides and ionically linked to chitosan;
a composition comprising cells encapsulated in alginate covalently linked to two or more different cell attachment peptides;
a composition comprising cells encapsulated in alginate mixed with two or more different cell attachment peptides;
a composition comprising cells encapsulated in alginate covalently linked to one or more cell attachment peptides and mixed with one or more cell attachment peptides;
a composition comprising cells encapsulated in alginate covalently linked to one cell attachment peptides and ionically linked to chitosan;
a composition comprising cells encapsulated in alginate covalently linked to two or more different cell attachment peptides and ionically linked to chitosan;
a composition comprising cells encapsulated in alginate mixed with one cell attachment peptides and ionically linked to chitosan;
a composition comprising cells encapsulated in alginate mixed with two or more different cell attachment peptides and ionically linked to chitosan
a composition comprising cells encapsulated in alginate covalently linked to one cell attachment peptide and mixed with cell attachment peptides and ionically linked to chitosan; and
a composition comprising cells encapsulated in alginate covalently linked to two or more different cell attachment peptide and mixed with cell attachment peptides and ionically linked to chitosan.

13. The system of claim 12 wherein said delivery device comprises two lumens.

14. The system of claim 12 wherein said delivery device comprises a syringe.

15. The system of claim 12 wherein the cell attachment peptides are selected from group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

16. The system of claim 12 wherein said composition comprises alginate beads.

17. The system of claim 16 wherein the beads have mean diameters in a range of from about 30 um to about 500 um.

18. The system of claim 12 wherein said composition comprises an alginate hydrogel.

19. The system of claim 12 wherein said composition comprises cells selected from the group consisting of: stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

20. The system of claim 12 wherein said composition further comprises one or more compounds selected from the group consisting of: EGF, VEGF, b-FGF, FGF, TGF, TGF-β and proteoglycans.

21. A system for repairing or treating a tissue or organ comprising a delivery device for providing two or more components to a tissue or organ, a first component comprising a gelling ion and a therapeutically effective amount of a second component selected from the group consisting of one or more of the following:
a composition comprising non-crosslinked alginate covalently linked to two or more different cell attachment peptides, and optionally further comprising cells;
a composition comprising non-crosslinked alginate mixed with two or more different cell attachment peptides and optionally further comprising cells; and
a composition comprising non-crosslinked alginate covalently linked to one or more cell attachment peptides and mixed with one or more cell attachment peptides and optionally further comprising cells.

22. The system of claim 21 wherein said delivery device comprises a first lumen for delivery of the first component and a second lumen for delivery of the second component.

23. The system of claim 21 wherein said delivery device comprises a syringe having two chambers, a first chamber for containing the first component and the second chamber for containing the second component.

24. The system of claim 21 wherein the cell attachment peptides are selected from group consisting of one or more of the following peptides: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

25. The system of claim 21 wherein said cells are selected from the group consisting of:
stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

26. A system for repairing or treating a tissue or organ comprising a delivery device for providing two or more components to a tissue or organ, a first component comprising a mixture of gelling ions and one or more cell attachment peptides; and a therapeutically effective amount of a second component selected from the group consisting of one or more of the following:
a composition comprising non-crosslinked alginate, and optionally further comprising cells;
a composition comprising non-crosslinked alginate covalently linked to one or more cell attachment peptides, and optionally further comprising cells;
a composition comprising non-crosslinked alginate mixed with one or more cell attachment peptides and optionally further comprising cells; and
a composition comprising non-crosslinked alginate covalently linked to one or more cell attachment peptides and mixed with one or more cell attachment peptides and optionally further comprising cells;
wherein said first component and said second component collectively comprise two or more different cell attachment peptides.

27. The system of claim 26 wherein said delivery device comprises a first lumen for delivery of the first component and a second lumen for delivery of the second component.

28. The system of claim 26 wherein said delivery device comprises a syringe having two chambers, a first chamber for containing the first component and the second chamber for containing the second component.

29. The system of claim 26 wherein the cell attachment peptides are selected from group consisting of one or more of the following peptides: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

30. The system of claim 26 wherein said cells are selected from the group consisting of: stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

31. A composition selected from the group consisting of:
a composition comprising alginate covalently linked to two or more different cell attachment peptides;
a composition comprising alginate mixed with two or more different cell attachment peptides;
a composition comprising alginate covalently linked to one or more cell attachment peptides and mixed with one or more cell attachment peptides;
a composition comprising alginate covalently linked to one cell attachment peptides and ionically linked to chitosan;
a composition comprising alginate covalently linked to two or more different cell attachment peptides and ionically linked to chitosan;
a composition comprising alginate mixed with one cell attachment peptides and ionically linked to chitosan;
a composition comprising alginate mixed with two or more different cell attachment peptides and ionically linked to chitosan
a composition comprising alginate covalently linked to one cell attachment peptide and mixed with cell attachment peptides and ionically linked to chitosan;
a composition comprising alginate covalently linked to two or more different cell attachment peptide and mixed with cell attachment peptides and ionically linked to chitosan;
a composition comprising cells encapsulated in alginate covalently linked to two or more different cell attachment peptides;
a composition comprising cells encapsulated in alginate mixed with two or more different cell attachment peptides;
a composition comprising cells encapsulated in alginate covalently linked to one or more cell attachment peptides and mixed with one or more cell attachment peptides;
a composition comprising cells encapsulated in alginate covalently linked to one cell attachment peptides and ionically linked to chitosan;
a composition comprising cells encapsulated in alginate covalently linked to two or more different cell attachment peptides and ionically linked to chitosan;
a composition comprising cells encapsulated in alginate mixed with one cell attachment peptides and ionically linked to chitosan;
a composition comprising cells encapsulated in alginate mixed with two or more different cell attachment peptides and ionically linked to chitosan
a composition comprising cells encapsulated in alginate covalently linked to one cell attachment peptide and mixed with cell attachment peptides and ionically linked to chitosan; and
a composition comprising cells encapsulated in alginate covalently linked to two or more different cell attachment peptide and mixed with cell attachment peptides and ionically linked to chitosan.

32. The composition of claim 31 wherein the cell attachment peptides are selected from group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

33. The composition of claim 31 wherein said composition comprises alginate beads.

34. The composition of claim 31 wherein the beads have mean diameters in a range of from about 30 um to about 500 um.

35. The composition of claim 31 wherein said composition comprises an alginate hydrogel.

36. The composition of claim 31 wherein said composition comprises cells selected from the group consisting of: stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

37. The composition of claim 31 wherein said composition further comprises one or more compounds selected from the group consisting of: EGF, VEGF, b-FGF, FGF, TGF, TGF-β and proteoglycans.

38. The composition of claim 31 wherein said composition is in a tissue selected from the group consisting of: bones, muscle, cartilage, connective, neural, epithelial, vascular, urothelial and mucosal.

39. The composition of claim 31 wherein said composition is in to an organ selected from the group consisting of: skin, sinus, liver, lungs, kidney, brain, pancreas, endocrine organs, esophagus, stomach, intestines, colon, rectum, cornea, and breast.

40. A method for delivering a desired protein to an individual comprising:
providing to the individual a therapeutically effective amount of a composition selected from the group consisting of one or more of the following:
a composition comprising cells encapsulated in alginate covalently linked to two or more different cell attachment peptides;
a composition comprising cells encapsulated in alginate mixed with two or more different cell attachment peptides;
a composition comprising cells encapsulated in alginate covalently linked to one or more cell attachment peptides and mixed with one or more cell attachment peptides;
a composition comprising cells encapsulated in alginate covalently linked to one cell attachment peptides and ionically linked to chitosan;
a composition comprising cells encapsulated in alginate covalently linked to two or more different cell attachment peptides and ionically linked to chitosan;
a composition comprising cells encapsulated in alginate mixed with one cell attachment peptides and ionically linked to chitosan;
a composition comprising cells encapsulated in alginate mixed with two or more different cell attachment peptides and ionically linked to chitosan
a composition comprising cells encapsulated in alginate covalently linked to one cell attachment peptide and mixed with cell attachment peptides and ionically linked to chitosan; and
a composition comprising cells encapsulated in alginate covalently linked to two or more different cell attachment peptide and mixed with cell attachment peptides and ionically linked to chitosan;
wherein said cells express the desired protein.

41. The method of claim 40 wherein the cell attachment peptides are selected from group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

42. The method of claim 40 wherein said composition comprises alginate beads.

43. The method of claim 42 wherein the beads have mean diameters in a range of from about 30 um to about 500 um.

44. The method of claim 40 wherein said composition comprises an alginate hydrogel.

45. The method of claim 40 wherein said composition comprises cells selected from the group consisting of: stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

46. The method of claim 40 wherein said composition further comprises one or more compounds selected from the group consisting of: EGF, VEGF, b-FGF, FGF, TGF, TGF-β and proteoglycans.

47. The method of claim 40 wherein said composition is provided as a cross-linked matrix.

48. The method of claim 40 wherein said composition produces a matrix in situ following application to the tissue or organ.

49. The method of claim 40 wherein said individual has been diagnosed with a disease or condition characterized by an absence or deficiency in a functional form of the desired protein.

50. A system for delivering a desired protein to an individual comprising a delivery device for providing a composition to a tissue or organ and a therapeutically effective amount of a composition selected from the group consisting of one or more of the following:
a composition comprising cells encapsulated in alginate covalently linked to two or more different cell attachment peptides;
a composition comprising cells encapsulated in alginate mixed with two or more different cell attachment peptides;
a composition comprising cells encapsulated in alginate covalently linked to one or more cell attachment peptides and mixed with one or more cell attachment peptides;
a composition comprising cells encapsulated in alginate covalently linked to one cell attachment peptides and ionically linked to chitosan;
a composition comprising cells encapsulated in alginate covalently linked to two or more different cell attachment peptides and ionically linked to chitosan;
a composition comprising cells encapsulated in alginate mixed with one cell attachment peptides and ionically linked to chitosan;
a composition comprising cells encapsulated in alginate mixed with two or more different cell attachment peptides and ionically linked to chitosan
a composition comprising cells encapsulated in alginate covalently linked to one cell attachment peptide and mixed with cell attachment peptides and ionically linked to chitosan; and
a composition comprising cells encapsulated in alginate covalently linked to two or more different cell attachment peptide and mixed with cell attachment peptides and ionically linked to chitosan;
wherein said cells express the desired protein.

51. The system of claim 50 wherein said delivery device comprises two lumens.

52. The system of claim 50 wherein said delivery device comprises a syringe.

53. The system of any of claims 50 wherein the cell attachment peptides are selected from group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

54. The system of claim 50 wherein said composition comprises alginate beads.

55. The system of claim 54 wherein the beads have mean diameters in a range of from about 30 um to about 500 um.

56. The system of claim 50 wherein said composition comprises an alginate hydrogel.

57. The system of claim 50 wherein said cells are selected from the group consisting of: stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

58. The system of claim 50 wherein said composition further comprises one or more compounds selected from the group consisting of: EGF, VEGF, b-FGF, FGF, TGF, TGF-β and proteoglycans.

59. A method of culturing cells in vitro comprising maintaining cells under conditions suitable for cell growth and proliferation in a composition selected from the group consisting of:
a composition comprising alginate covalently linked to two or more different cell attachment peptides;
a composition comprising alginate mixed with two or more different cell attachment peptides;
a composition comprising alginate covalently linked to one or more cell attachment peptides and mixed with one or more cell attachment peptides;
a composition comprising alginate covalently linked to one cell attachment peptides and ionically linked to chitosan;
a composition comprising alginate covalently linked to two or more different cell attachment peptides and ionically linked to chitosan;
a composition comprising alginate mixed with one cell attachment peptides and ionically linked to chitosan;
a composition comprising alginate mixed with two or more different cell attachment peptides and ionically linked to chitosan
a composition comprising alginate covalently linked to one cell attachment peptide and mixed with cell attachment peptides and ionically linked to chitosan; and
a composition comprising alginate covalently linked to two or more different cell attachment peptide and mixed with cell attachment peptides and ionically linked to chitosan.

60. The method of claim 59 wherein the cell attachment peptides are selected from group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22).

61. The method of claim 59 wherein said composition comprises alginate beads.

62. The method of claim 61 wherein the beads have mean diameters in a range of from about 30 um to about 500 um.

63. The method of claim 59 wherein said composition comprises an alginate hydrogel.

64. The method of claim 59 wherein said composition comprises cells selected from the group consisting of: stem cells, muscle cells, pancreatic islets, chondrocytes, hepatic cells, neural cells, renal cortex cells, vascular endothelial cells, thyroid and parathyroid cells, adrenal cells, thymic cells, ovarian cells, and cells which include recombinant genetic material provided to express desired proteins.

65. The method of claim 59 wherein said composition further comprises one or more compounds selected from the group consisting of: EGF, VEGF, b-FGF, FGF, TGF, TGF-β and proteoglycans.


SEQUENCE LISTING (22 sequences)

SEQ ID NO: 1 - protein, 5 aa, Artificial Sequence
  Cell Adhesion Peptide 1
YIGSR

SEQ ID NO: 2 - protein, 5 aa, Artificial Sequence
  Cell Adhesion Peptide 2
IKVAV

SEQ ID NO: 3 - protein, 4 aa, Artificial Sequence
  Cell Adhesion Peptide 3
REDV

SEQ ID NO: 4 - protein, 4 aa, Artificial Sequence
  Cell Adhesion Peptide 4
DGEA

SEQ ID NO: 5 - protein, 6 aa, Artificial Sequence
  Cell Adhesion Peptide 5
VGVAPG

SEQ ID NO: 6 - protein, 5 aa, Artificial Sequence
  Cell Adhesion Peptide 6
GRGDS

SEQ ID NO: 7 - protein, 4 aa, Artificial Sequence
  Cell Adhesion Peptide 7
RGDV

SEQ ID NO: 8 - protein, 5 aa, Artificial Sequence
  Cell Adhesion Peptide 8
PDSGR

SEQ ID NO: 9 - protein, 7 aa, Artificial Sequence
  Cell Adhesion Peptide 9
RYVVLPR

SEQ ID NO: 10 - protein, 6 aa, Artificial Sequence
  Cell Adhesion Peptide 10
LGTIPG

SEQ ID NO: 11 - protein, 4 aa, Artificial Sequence
  Cell Adhesion Peptide 11
RGDS

SEQ ID NO: 12 - protein, 4 aa, Artificial Sequence
  Cell Adhesion Peptide 12
RGDF

SEQ ID NO: 13 - protein, 12 aa, Artificial Sequence
  Cell Adhesion Peptide 13
HHLGGALQAGDV

SEQ ID NO: 14 - protein, 4 aa, Artificial Sequence
  Cell Adhesion Peptide 14
VTCG

SEQ ID NO: 15 - protein, 4 aa, Artificial Sequence
  Cell Adhesion Peptide 15
SDGD

SEQ ID NO: 16 - protein, 6 aa, Artificial Sequence
  Cell Adhesion Peptide 16
GREDVY

SEQ ID NO: 17 - protein, 5 aa, Artificial Sequence
  Cell Adhesion Peptide 17
GRGDY

SEQ ID NO: 18 - protein, 6 aa, Artificial Sequence
  Cell Adhesion Peptide 18
GRGDSP

SEQ ID NO: 19 - protein, 4 aa, Artificial Sequence
  Cell Adhesion Peptide 19
VAPG

SEQ ID NO: 20 - protein, 9 aa, Artificial Sequence
  Cell Adhesion Peptide 20
GGGGRGDSP

SEQ ID NO: 21 - protein, 8 aa, Artificial Sequence
  Cell Adhesion Peptide 21
GGGGRGDY

SEQ ID NO: 22 - protein, 6 aa, Artificial Sequence
  Cell Adhesion Peptide 22
FTLCFD